(19) United States
(12) Patent Application Publication
(10) Pub. No.: US 2025/0242120 A1
(43) Pub. Date: Jul. 31, 2025

(54) CLINICAL DATA CORRELATION WITH ACOUSTIC REFLECTOMETRY DATA

(71) Applicant: Covidien LP, Mansfield, MA (US)

(72) Inventors: Jeffrey P. Mansfield, Loveland, CO (US); Deborah Blundell, Denver, CO (US); Kristen Anne Hood, Denver, CO (US); Matthew J Phillips, Carlsbad, CA (US); Abigail Catherine Scaggs, Denver, CO (US)

(73) Assignee: Covidien LP, Mansfield, MA (US)

(21) Appl. No.: 18/989,570

(22) Filed: Dec. 20, 2024

Related U.S. Application Data
(60) Provisional application No. 63/625,142, filed on Jan. 25, 2024.

(51) Int. Cl. A61M 16/04; A61M 16/00; G16H 10/60

(52) U.S. Cl. CPC A61M 16/0402; A61M 16/0003; A61M 16/024; G16H 10/60; A61M 2205/3303; A61M 2205/3306; A61M 2205/3327; A61M 2205/3331; A61M 2205/3375; A61M 2210/1032; A61M 2230/04; A61M 2230/20; A61M 2230/42; A61M 2230/43

(57) ABSTRACT

The technology relates to acoustic reflectometry systems and methods. An example system includes an acoustic sensor that includes a generator that emits acoustic pulses; and a sensor that detects the emitted acoustic pulses and reflections thereof. The system further includes a monitor, in communication with the acoustic sensor. The monitor performs operations including: receive patient data for a current patient receiving ventilation; provide the patient data as input to a trained clinical model; receive, as output from the trained clinical model, a clinically relevant threshold for an acoustically measurable parameter; process the detected incident and reflected waves to determine a value of the acoustically measurable parameter; compare the value of the acoustically measurable parameter to the clinically relevant threshold; and based on the comparison, generate a notification.

### CROSS REFERENCE TO RELATED APPLICATIONS

This application claims the benefit of U.S. Provisional Application No. 63/625,142 filed Jan. 25, 2024, entitled “Clinical Data Correlation with Acoustic Reflectometry Data,” which is incorporated herein by reference in its entirety.

### BACKGROUND

Medical ventilators offer critical support for patients who require ongoing breathing assistance. An endotracheal tube (ETT) is often used in conjunction with a ventilator, as part of a breathing circuit. The ETT is placed, via intubation, through patient's mouth into the trachea, bypassing the upper airway. A number of sensors may be used with the ETT to ensure proper positioning within the trachea and to ensure that the patient is receiving adequate ventilation. Measurement data collected by the sensors may be correlated and may provide further information for monitoring the patient's respiratory condition. It is with respect to this general technical environment that aspects of the present technology disclosed herein have been contemplated. Furthermore, although a general environment is discussed, it should be understood that the examples described herein should not be limited to the general environment identified herein.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter. Additional aspects, features, and/or advantages of examples will be set forth in part in the description which follows and, in part, will be apparent from the description, or may be learned by practice of the disclosure.

In an aspect, the technology relates to an acoustic reflectometry system. The system includes an acoustic sensor that includes a generator that emits acoustic pulses; and a sensor that detects the emitted acoustic pulses and reflections thereof. The system further includes a monitor, in communication with the acoustic sensor. The monitor includes a processor; and memory storing instructions that, when executed by the processor, causes the acoustic monitor to perform operations. The operations include receive patient data for a current patient receiving ventilation; provide the patient data as input to a trained clinical model; receive, as output from the trained clinical model, a clinically relevant threshold for an acoustically measurable parameter; process the detected incident and reflected waves to determine a value of the acoustically measurable parameter; compare the value of the acoustically measurable parameter to the clinically relevant threshold; and based on the comparison, generate a notification.

In an example, the patient data includes at least one of age, height, weight, or a known medical condition. In another example, the acoustically measurable parameter is one of tracheal tube movement, tracheal tube position, obstruction size, or passageway size. In another example, the operations further include receiving patient sensor data; and providing the patient sensor data as input to the one or more trained clinical models, wherein the clinically relevant threshold is further based on the patient sensor data. In a further example, the patient sensor data includes at least one of capnometry data, pulse oximetry data, or electrocardiogram data. In still another example, the operations further include receiving ventilator data; and providing the ventilator data as input to the one or more trained clinical models, wherein the clinically relevant threshold is further based on the ventilator data. In a further example, the ventilator data includes at least one of a current ventilation mode, a ventilation setting, a pressure value, a flow value, a respiratory rate, work of breathing, indication of patient-initiated spontaneous breaths.

In another aspect, the technology relates to a method for detecting clinical conditions with an acoustic system. The method includes receiving acoustic data from an acoustic sensor coupled to a tracheal tube of a patient being ventilated; receiving patient demographic data for the patient; receiving patient sensor data; processing the acoustic data to determine a value for an acoustically measurable parameter; based on at least the patient demographic data, receiving from a clinical model a predicted correlation between the patient sensor data and the acoustically measurable parameter; based on the value of the acoustically measurable parameter and the patient sensor data, determining that the patient sensor data and the acoustically measurable parameter diverge from the predicted correlation; and based on the divergence, adjusting a threshold for the acoustically measurable parameter.

In an example, the acoustically measurable parameter is one of tracheal tube movement, tracheal tube position, obstruction size, or passageway size. In another example, the acoustically measurable parameter is obstruction size and the patient sensor data is pulse oximetry data. In a further example, the divergence indicates that patient is more sensitive to obstruction size, and adjusting the threshold includes reducing the threshold. In yet another example, the patient sensor data is at least one of capnometry data, pulse oximetry data, or electrocardiogram data. In still another example, the method further includes providing the patient demographic data as input to the clinical model; and receiving, based on processing of the patient demographic data by the clinical model, an initial clinically relevant threshold for the acoustically measurable parameter, wherein adjusting the threshold includes adjusting the initial clinically relevant threshold. In yet another example, the method further includes determining that a subsequent value for the acoustically measurable parameter exceeds the adjusted threshold; and generating a notification based on determining that the value exceeds the adjusted threshold. In still yet another example, the patient demographic data includes at least one of age, height, weight, or a known medical condition.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIG. 1 depicts an example airway management system.

FIG. 2A depicts an example acoustic sensor package.

FIG. 2B depicts a view of the front panel of a monitor associated with the acoustic sensor package of FIG. 2A.

FIGS. 2C-D depict examples of monitor display content.

FIG. 3 depicts an example of a network-connected system in which patient systems receive clinically relevant alert thresholds from a host system.

FIG. 4 depicts an example method for receiving and using clinically relevant alert thresholds.

FIG. 5 depicts an example method for receiving, updating, and using clinically relevant alert thresholds.

### DETAILED DESCRIPTION

An endotracheal tube (ETT) is a type of breathing tube that is placed through the nose or mouth of a patient and into the trachea. The proximal end of the tube remains outside the patient and is typically connected to a mechanical ventilator to form a patient breathing circuit. The ETT helps maintain patency of the airway, permits positive pressure ventilation; seals off the digestive tract from the trachea (thereby preventing gastric insufflation due to forced air into the stomach), and provides a means for supplying oxygen, anesthesia, or other breathing gases to a patient requiring respiratory support. An ETT is placed in the patient's airway by a clinician through a process called intubation. Once placed, the ETT may be affixed to the patient by medical tape, elastic harness, or other method, in order to help the distal end of the ETT remain in position in the trachea.

Despite action by a clinician to help keep an ETT in place in the trachea, the position of the ETT may drift over time, such that the distal tip of the ETT may relocate to a portion of the patient's anatomy that may reduce ventilatory efficacy or present a health or safety risk. For example, the ETT may drift inferiorly, towards or into the left or right mainstem bronchi, or into deeper branches of the bronchial tree. In other examples, the ETT may drift superiorly, towards or above the larynx. In examples where the ETT drifts superiorly, the dislocation of the ETT may be so significant that a clinician is required to perform an extubation, followed by a re-intubation, in order to position the ETT correctly. In similar examples, the ETT may be completely expelled by the patient, requiring re-intubation. This drift in ETT position may be especially problematic for pediatric patients, such as neonatal patients, preterm infants, or infants requiring intensive care, such as in a neonatal intensive care unit (NICU). Unplanned extubations are a sadly common adverse event affecting mechanically ventilated NICU patients (impacting nearly 1 in 5 patients) and may result in injury and/or significant increases in hospital costs and hospital stays.

One approach for monitoring the position of the distal tip of the ETT and detecting ETT obstructions is to use an acoustic reflectometry device. One such device is described in U.S. Pat. No. 10,668,240, titled “Acoustical Guidance and Monitoring System,” (hereinafter “the '240 patent”) which is incorporated herein by reference in its entirety. Briefly, the acoustic reflectometry device is connected to the proximal end of the ETT, between the ETT and tubing associated with a ventilator. The device uses a sound pulse generator to transmit acoustic waves (e.g., pulses) from the proximal tip of the ETT towards the distal tip. For instance, the input waveforms can be pulses or other time-limited waveforms. The pulses may consist of any time-limited waveform. These pulses interact with the distal tip of the tube and the patient's anatomy, and the acoustic pulses are reflected back to the device, which also houses two or more sound-sensing elements (e.g., microphones, acoustic receivers). The received acoustic reflections are processed and analyzed to determine positional drift of the ETT distal tip relative to a baseline position. The received reflections may also be analyzed to determine whether the distal tip of the ETT has entered a larger or smaller diameter respiratory passageway. Obstructions or blockages within the ETT may also be located and characterized based on the reflections of the acoustic pulses. The position and/or blockage information derived from the acoustic analysis is displayed on a monitor associated with the device.

In some examples, the position and blockage information may be compared to a set of alert thresholds. If one or more of the alert thresholds is exceeded, the monitor may provide a notification to a clinician. The clinician may then address positional shifts or blockages in the ETT to help prevent unplanned extubation or address obstructions within the ETT.

The acoustic device may also be used for detecting airway collapse or airway constrictions in the vicinity of the ETT distal tip. For example, certain respiratory conditions may cause a total or partial collapse of the airway during a portion of an exhalation due to an inability of the patient's respiratory system to maintain sufficient airway pressure. Analysis of the acoustic reflections may indicate a change in the diameter of the airway in the vicinity of the ETT distal tip, relative to a baseline diameter. If the diameter of the airway drops below a specified alert threshold, the device may provide a notification. In response, the clinician may change one or more ventilation parameters to prevent the airway constriction. For instance, following detection of airway constriction, a clinician may increase the positive end-expiratory pressure (PEEP) setting of the ventilator so that airway pressure is maintained near the end of exhalation.

The values or parameters that can be measured by the acoustic reflectometry system may be referred to as acoustically measurable parameters, and may include parameters such as ETT position, ETT movement, ETT obstruction position and/or size, airway constriction or expansion (e.g., airway size directly distal to the ETT, passageways such as esophagus, bronchus, trachea, upper airway, mouth), among other parameters measurable by the acoustic reflectometry system.

The above alert thresholds for ETT position, ETT obstruction, and airway diameter (among other possible alert thresholds) may be provided by the clinician. However, in some examples, the provided alert thresholds may not be correlated with a significant health risk. In examples, alert thresholds may be provided based on default threshold settings, common practice, generalized recommendations, or other factors that may not be correlated with a demonstrated clinical health risk. For instance, a default alert threshold for a 30% obstruction of the ETT may be set for the system, and in many cases this default setting is unchanged by the clinician. The clinical effect of obstructions within the ETT tube, however, may differ between different patient types. For some patients a 30% obstruction may affect respiration, such as by decreasing blood oxygen saturation (SpO₂), changing end-tidal CO₂ (EtCO₂), and/or affecting other measurable respiratory metrics. For other patients, a 30% obstruction may have minimal impact on SpO₂, EtCO₂, and/or other measures of respiration. Providing a notification to a clinician for an ETT condition that poses minimal health risk to a patient may result in unnecessary interaction with the ETT. For some patients, such as neonatal patients or other sensitive patient groups, interaction with the ETT (e.g., adjusting the ETT position, performing suction, etc.) may itself pose a health risk to the patient, due to the small size of the airway, the condition of the patient, and/or other factors. Thus, it may be beneficial to reduce clinician interaction with the ETT to the extent possible.

The present technology provides systems and methods through which clinically relevant alert thresholds are provided to a monitor, which uses the alert thresholds to issue notifications to the clinician when a condition of the ETT may pose a health risk to the patient. The clinically relevant alert thresholds are provided by one or more clinical models that are trained using patient population data. The clinical models incorporate correlations between clinical data (e.g., sensor data), patient demographic data, and acoustic reflectometry data determined during model training. When trained, the clinical models receive data from a monitor and provide clinically relevant thresholds associated with ETT position, ETT obstruction, airway constriction, and/or other conditions associated with the ETT. Additional details are provided below, with respect to the accompanying drawings.

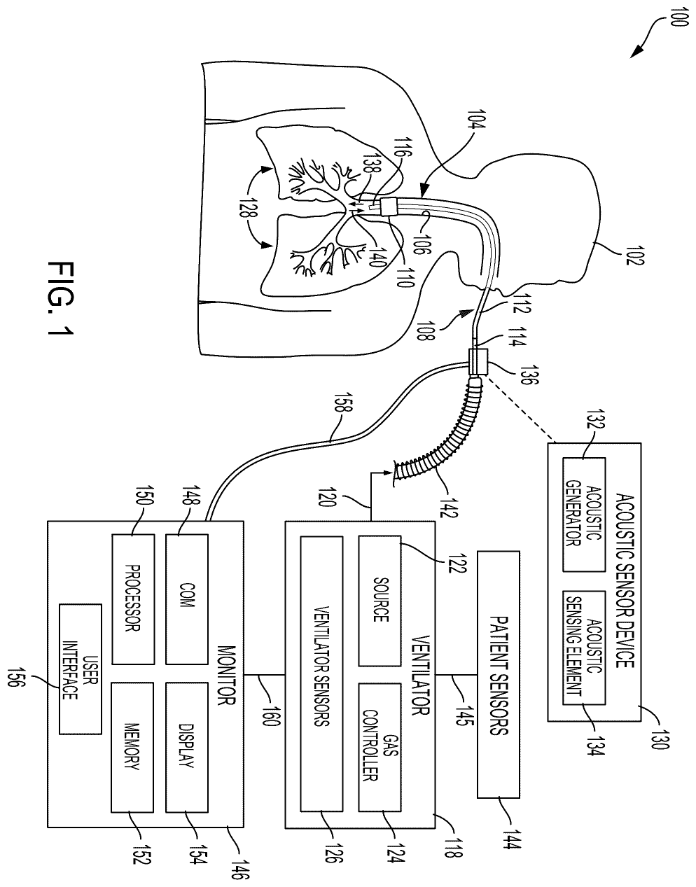

FIG. 1 depicts an example of an airway management system or medical ventilation system 100 that includes a ventilator 118, a monitor 146, an acoustic sensor package or device 130, and a tracheal tube 108 (e.g., ETT). The tracheal tube 108 is illustrated as an endotracheal tube, which has an inflatable balloon cuff 110 that may be inflated to form a seal against walls 106 of a trachea 104 of a patient 102. However, the tracheal tube 108 may alternatively be uncuffed. The airway management system 100 may be used in conjunction with any other suitable types of tracheal tubes or medical devices. As examples, the airway management system 100 may be utilized with an endotracheal tube, an endobronchial tube, a tracheostomy tube, an introducer, an endoscope, a bougie, a circuit, an airway accessory, a connector, an adapter, a filter, a humidifier, nasal cannula, or a supraglottic mask/tube.

The system 100 includes devices that facilitate positive pressure ventilation of the patient 102, such as the ventilator 118, which provides mechanical ventilation to the patient 102. For example, the ventilator 118 may provide a gas mixture 120 (e.g., from a source 122 of the gas mixture 120) through the acoustic sensor device 130, through the tracheal tube 108, and to lungs 128 of the patient 102, thereby mechanically actuating rest, inspiration, and expiration phases of breathing cycles of the patient 102. The gas mixture 120 may be referred to herein as breathing gases. In some examples, the ventilator 118 includes a gas mixture controller 124 that provides control instructions to cause the ventilator 118 to continuously or intermittently adjust a pressure and/or a composition of the gas mixture 120 provided from the source 122 and to the patient 102. For example, the gas mixture controller 124 may cause the ventilator 118 to direct air, oxygen, or another suitable gas mixture from the source 122 to the lungs 128 of the patient 102.

The acoustic sensor device 130 is connected to the ventilator 118 through a patient circuit 142, which is depicted as a single-limb circuit (e.g., an inhalation limb) used for providing the gas mixture 120 to the patient 102 for inhalation. In examples where the cuff 110 is not included or is not inflated, the patient 102 may exhale through the nose or mouth (if applicable). In examples where the cuff 110 is inflated, the patient 102 may exhale through the tracheal tube 108, and the exhaled breathing gases may be vented through an exhaust port (not depicted) provided as part of the patient circuit 142. In other examples, the patient circuit 142 may be a dual-limb circuit, where additional tubing is provided for venting exhaled gases from the patient 102 (e.g., an exhalation limb). The exhalation limb may be connected to the ventilator 118, which may provide elements for controlling the exhaled breathing gases. For example, the ventilator 118 may include one or more valves, filters, and/or other elements used for managing the flow of exhaled breathing gases. In some examples, to support exhalation, another type of tracheal tube 108 (or other device) may be used in place of the depicted tracheal tube 108 (e.g., one of the tracheal tube alternatives described above).

The ventilator 118 may include a plurality of ventilator sensors 126 that provide measurement data associated with the gas mixture 120. The ventilator sensors 126 may be internal to the ventilator 118 and/or may be coupled to the patient circuit 142. For example, one or more ventilator sensors 126 may provide measurement data associated with flow, pressure, respiratory rate (RR), work of breathing, indications of patient-initiated spontaneous breaths, and/or other respiratory parameters of breathing gases delivered to the patient 102 during inhalation. Additional data may be derived from the measured sensor data. For example, the volume of the delivered gas mixture 120 may be computed using flow measurement data, such as by performing integration of the measured flow data over time. Accordingly, the ventilator 118 may be capable of determining and reporting inhalation volumes (e.g., tidal volume (V_{T})) derived from measurement data acquired by the ventilator sensors 126. The ventilator may also generate and/or provide data regarding current ventilation settings, such as ventilation mode, positive end-expiratory pressure (PEEP) settings, fraction of inspired oxygen (FiO₂) settings, among other types of settings.

In examples where the tracheal tube 108, patient circuit 142, and ventilator 118 are configured to receive exhaled breathing gases from the patient 102, the ventilator sensors 126 may include additional pressure, flow, and/or other types of sensors for collecting measurements of the exhaled breathing gases. The ventilator 118 may further be capable of determining exhaled breathing volumes as described above.

The airway management system 100 may also include one or more patient sensors 144 that may be directly or indirectly connected to the patient 102 for collecting additional measurement data. For example, the patient sensors 144 may include a type of pulse oximeter, which provides a noninvasive measurement of oxygen saturation in the patient's blood (e.g., SpO₂). The pulse oximeter may also provide a measurement of the heart rate (HR) of the patient 102 or, in some examples, the patient sensors 144 may include a separate sensor for measuring HR and other information, such as electrocardiogram (ECG) data. The sensor data may also or alternatively be communicated to system components other than the ventilator, such as a multi-parameter monitor (MPM).

In another example, the patient sensors 144 may include a sensor or sensing system for measuring the CO₂ concentration of the exhaled breathing gases. For instance, the patient sensors 144 may include a capnometry monitor or sensor that samples a portion of the exhaled breathing gases and determines CO₂ concentration. In one example, the capnometry monitor may sample exhaled breathing gases via a port provided on the acoustic sensor device 130 or patient circuit 142. The capnometry monitor may provide a measurement of the EtCO₂.

Additionally or alternatively, the patient sensors 144 may include other types of sensors for collecting measurements from the patient 102. The patient sensors 144 may provide the measured data to the ventilator 118 via one or more sensor cables 145. Sensor data and other information may be transmitted between the ventilator 118 and patient sensors 144 via the sensor cable(s) 145 in analog or digital form, and the transmission may be performed according to any of a wide variety of communications protocols. In some examples, the sensor data may be provided by one or more of the patient sensors 144 to the ventilator 118 via wireless protocols, such as Wi-Fi, Bluetooth, or other established or custom wireless transceiver protocol.

In still other examples, the ventilator 118 may include the elements, functions, and/or features for implementing some or all of the measurements performed by the patient sensors 144. In one example, the ventilator 118 may include functions/elements for performing capnometry within the ventilator 118 itself. In other examples, the ventilator 118 may include functions/elements for performing SpO₂, HR, and/or other types of measurements.

Additionally or alternatively, one or more of the patient sensors 144 may be connected to the monitor 146, rather than the ventilator 118. For example, the patient sensors 144 may include a pulse oximeter that provides SpO₂ data directly to the monitor 146. In further examples, the monitor 146 may be connected to a capnometry monitor or may include the functions/elements for performing capnometry within the monitor 146.

As illustrated, the acoustic sensor device 130 of the airway management system 100 is coupled to an external or proximal end 114 of the tracheal tube 108. In the illustrated example, the acoustic sensor device 130 may operate as, or be, an adapter that facilitates coupling of the tracheal tube 108 to a patient circuit 142 or hose/tube coupled to the ventilator 118. Other arrangements are also contemplated, such as an acoustic sensor device 130 that is disposed on the tracheal tube 108 or on other components of the breathing circuit.

The acoustic sensor device 130 includes at least one acoustic generator 132 and at least one acoustic sensor 134 disposed within an adapter housing 136. The acoustic generator 132 is oriented to direct and/or emit incident sound energy 138 (e.g., acoustic pulses, sound, acoustic energy) into the lumen 112 of the tracheal tube 108, which guides the sound energy 138 out of an internal or distal end 116 of the tracheal tube 108 and toward airways of lungs 128 of the patient 102. Further, the acoustic sensor 134 detects reflected sound energy 140 (e.g., detects reflections of the emitted acoustic pulses) or echoes of the incident sound energy 138, back from different positions along the endotracheal tube/and or the airways of the patient. For instance, the emitted acoustic pulses may reflect from items such as obstructions in the tracheal tube, the tip of the tracheal tube, the airways of the lungs 128, and/or other passageways of the body. Accordingly, the acoustic sensor device 130 facilitates acoustic reflectometry techniques that analyze sound pressure waveforms for airway acoustic echoes indicative of airway size. That is, the acoustic generator 132 and the acoustic sensor 134 cooperate to provide sensor signals indicative of a sound pressure waveform having an airway acoustic echo, which the monitor 146 may analyze to determine, among other things, an airway size of the trachea around, or distally located from, the tip of the tracheal tube 108.

The acoustic generator 132 in some examples is a speaker or a miniature speaker. However, the acoustic generator 132 may additionally or alternatively include any suitable loudspeakers, buzzers, horns, sounders, and so forth that rely on moving coil, electrostatic, isodynamic, or piezo-electric techniques. Additionally, the sound sensing element(s) 134 (also referred to herein as an acoustic sensor 134) may be a microphone, microphone array, or other sound pressure sensors, in some examples. When implemented as a microphone array, the acoustic sensor 134 and/or the monitor 146 discussed below may be designed to separate the input sound pulses (e.g., distally travelling sound waves) from the output sound pulses (e.g., proximally traveling sound waves). The input sound waves (X) and the output sound waves (Y) may be used to calculate an impulse response (H) of the ETT and the airways (e.g., H=Y/X). Thus, the use of the microphone array allows for taking not only the incident pulse as the input signal, but all of the extraneous echoes from the ventilator side and leveraging them as input energy to the system as well. Further, when implemented as a microphone array, the acoustic sensor 134 may be designed to determine the speed of travel or propagation (sound speed) of the emitted or reflected acoustic pulses in the region between sound sensing elements within the microphone array, or in the region between the acoustic generator 132 and one or more of the sound sensing elements. Other suitably paired components that respectively generate suitable sound energy and receive echoes or reflection of the sound energy may be used in the acoustic sensor device 130. Additional details regarding acoustic reflectometry are described in U.S. Pat. No. 9,707,363, titled “System and Method for Use of Acoustic Reflectometry Information in Ventilation Devices,” which is incorporated herein by reference in its entirety, and the '240 patent, referenced above. For instance, example components for the acoustic sensor(s) 134 and the acoustic generator 132 are described in the '240 patent.

In some examples, the acoustic sensor device 130 may not include an acoustic generator 132, or the acoustic generator 132 may be permanently or intermittently left unpowered. In such examples, one or more sound-sensitive elements of acoustic sensor 134 may acquire sounds generated naturally by the body of the patient 102. Analysis of these sounds may provide relevant data or information about the health of the patient 102. For example, analysis of naturally occurring, internal body sounds may indicate the presence or onset of congestion, wheezing, pneumonia, and/or other health condition.

In other examples, when no acoustic pulses are coupled/directed into the lumen 112 by an acoustic generator 132, the acoustic sensor 134 may detect transmitted and/or reflected acoustic energy generated by other sources. For example, the ventilator 118 may inherently generate acoustic energy that couples into the lumen 112 of the tracheal tube 108, and may be suitable for performing some level of acoustic reflectometry, as described above. In some examples, other acoustic sources may provide suitable acoustic energy for performing acoustic reflectometry, such as sources that may be coupled to the patient circuit 142. Acquisition and analysis of acoustic energy provided by sources other than the acoustic generator 132 may include the use of specific filtering and/or data processing that differs from configurations where the acoustic generator 132 is the source of acoustic energy.

The acoustic sensor device 130 may be communicatively coupled to the monitor 146 via interface cable 158. The interface cable 158 provides a means for acoustic measurement and other data to be transmitted from the acoustic sensor device 130 to the monitor 146. The transmitted data includes acoustic measurement data acquired by the acoustic sensor 134 (such as the types of data described above) that has been processed by elements of the acoustic sensor device 130. For example, the transmitted acoustic measurement data may include data that has been amplified, filtered, digitized (such as by an analog-to-digital converter (ADC)), and/or that has otherwise been processed. In some examples, the acoustic sensor device 130 may transmit unprocessed, or minimally processed, acoustic measurement data. For instance, the acoustic sensor device 130 may transmit the signals acquired by the acoustic sensor 134 in a relatively unprocessed state, such as with minimal (if any) amplification and/or filtering. The acoustic sensor device 130 may also transmit other types of data to the monitor 146 by way of the interface cable 158. For example, elements within the acoustic sensor device 130 may transmit status, information related to error conditions, device settings, configuration information, calibration information, or other types of information.

Additionally, the monitor 146 may transmit information to the acoustic sensor device 130 via interface cable 158. For instance, the monitor 146 may transmit sensor operating parameters, settings, data, variables, firmware, executable code, or other information or data associated with operation of acoustic sensor device 130. The interface cable 158 may also provide a means for the monitor 146 to provide electrical power to the acoustic sensor device 130. In examples, the interface cable 158 may be implemented as two or more cables, wires, or other type of suitable electrical conductor.

The monitor 146 may include a communication connection(s) 148 (e.g., input/output ports, communication circuitry, analog and/or interface circuitry, etc.), processing circuitry, such as the processor 150, memory 152, a display 154, and a user interface 156 that may be communicatively coupled to one another. The communication connection(s) 148 may receive data from, and/or transmit data to, the acoustic sensor device 130 via interface cable 158. Data may be transmitted between the communication connection(s) 148 and the sensor device 130 in analog or digital form, and the transmission may be performed according to any of a wide variety of communications protocols. In other examples, the communication connection(s) 148 may communicate with the acoustic sensor device 130 via wireless protocols, such as Wi-Fi, Bluetooth, or other established or custom wireless transceiver protocol.

In further examples, the communication connection(s) 148 may receive data from, and/or transmit data to, the ventilator 118 via ventilator cable 160. For instance, the ventilator 118 may provide data from ventilator sensors 126 and/or patient sensors 144 to the monitor 146. The ventilator 118 may also provide patient demographic data to the monitor 146, such as the age, gender, height, weight, known medical conditions, and/or other types of patient-specific data. The sensor data and patient demographic data may be used by the monitor 146 as described below.

The communication connection(s) 148 may also provide data to the ventilator 118 from the monitor 146. For example, the monitor 146 may provide acoustic sensor data to the ventilator 118 and/or may provide other types of data to the ventilator 118, via the ventilator cable 160. As another example, the monitor 146 may provide signals for controlling/adjusting ventilator settings to the ventilator 118.

In some examples, the communication connection(s) 148 may communicate with the ventilator 118 via wireless protocols, such as Wi-Fi, Bluetooth, or other established or custom wireless transceiver protocol. In such examples, the ventilator cable 160 may not be present in the system 100.

The monitor 146 includes processing circuitry, such as processor 150, which may include any combination of digital and analog circuitry needed for processing signals received from, or transmitted to, the acoustic sensor device 130. Examples of analog processing circuitry may include signal filters, amplifiers, bridge circuits, bias circuits, pulse generators, level translators, ADCs, or other types or combinations of analog circuitry. The processor 150 may include one or more general purpose processors, microprocessors, microcontrollers, digital signal processors (DSPs), graphics processing units (GPUs), or other programmable circuits. In examples, the processor 150 may include any combination of commercially available components, or custom or semi-custom integrated circuits, such as application specific integrated circuits (ASICs). The processor 150 may include elements needed for control or communication with the communication connection(s) 148, memory 152, display 154, user interface 156, and/or other elements of the monitor 146.

The processor 150 may perform control, interface, communication, or other processing functions by executing instructions that are stored in the memory 152. The memory 152 may include RAM, ROM, electrically erasable programmable read-only memory (EEPROM), flash memory or other memory technology, CD-ROM, digital versatile disks (DVD) or other optical storage, magnetic cassettes, magnetic tape, magnetic disk storage or other magnetic storage devices, or other types of storage media. The memory 152 may store instructions, that when executed by the processor 150, cause the monitor 146 or elements thereof to perform the operations described herein. The memory 152 may be used to store data, such as data acquired directly from the acoustic sensor device 130 and/or processed by the processor 150. The memory 152 may be used to store data acquired by other sensors (e.g., patient sensors 144 and/or ventilator sensors 126), patient demographic data, settings, alert thresholds, preferences, or other types of data or information used during operation of the monitor 146.

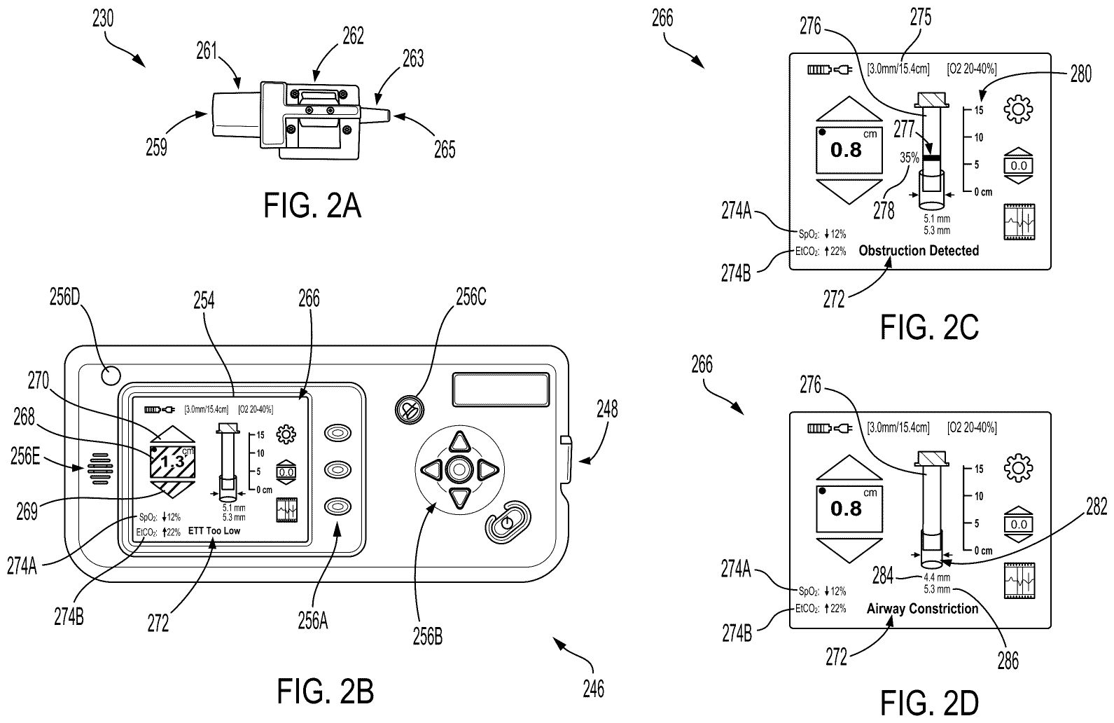

The processor 150, and/or other elements of the monitor 146, may receive user input from a user interface 156. In examples, the user interface 156 may include buttons, knobs, soft keys, dials, switches, and/or other forms of user-selectable input that may be made available to the user on the exterior of the monitor 146 (depicted in FIG. 2A). The user interface 156 may allow a clinician to configure the operation of the monitor 146 and/or the acoustic sensor device 130. For example, the user interface 156 may allow a clinician to enable/disable features and functions of the monitor 146 and/or acoustic sensor device 130, set alert thresholds, respond to alerts, navigate menus, configure settings, store/retrieve measurement data to or from memory 152, and/or provide other types of input to the monitor 146, among other functions. In further examples, the user interface 156 may allow a clinician to enable automatic features of the monitor 146, such as an automatic threshold adjustment feature as described below.

In some examples, the user interface 156 may provide audible alerts, such as through a speaker coupled to the user interface 156 (depicted in FIG. 2A). For example, the monitor 146 may be configured to provide an audible alert when the monitor detects a clinically relevant airway constriction, obstruction of the tracheal tube 108, and/or for other conditions that require the attention of a clinician. In other examples, the user interface 156 may provide audible alerts for any of a range of possible states and conditions associated with the operation of the monitor 146, acoustic sensor device 130, and/or other portion of the example airway management system 100.

The monitor 146 also includes a display 154, which may display textual, graphical, and/or other forms of displayable information or data. The information/data may be associated with position of the tracheal tube 108, obstructions within the tracheal tube 108, airway collapse, insufficient ventilation of the patient 102, information related to the respiratory status of the patient 102, data collected or derived from the sensors (e.g., ventilator sensors 126, patient sensors 144, etc.), other configurable settings, alerts, and/or other information associated with the functioning and operation of the monitor 146. The display 154 may be any of a variety of display technologies, such as liquid crystal display (LCD), light emitting diode (LED), organic light emitting diode (OLED), or other display technology. In examples, the display 154 may be a touch-sensitive display (e.g., a capacitive touch-sensitive display) that allows the clinician to provide input through the display 154, such as through a graphical user interface (GUI) provided on the display 154 by the user interface 156. An example GUI is depicted in FIG. 2A.

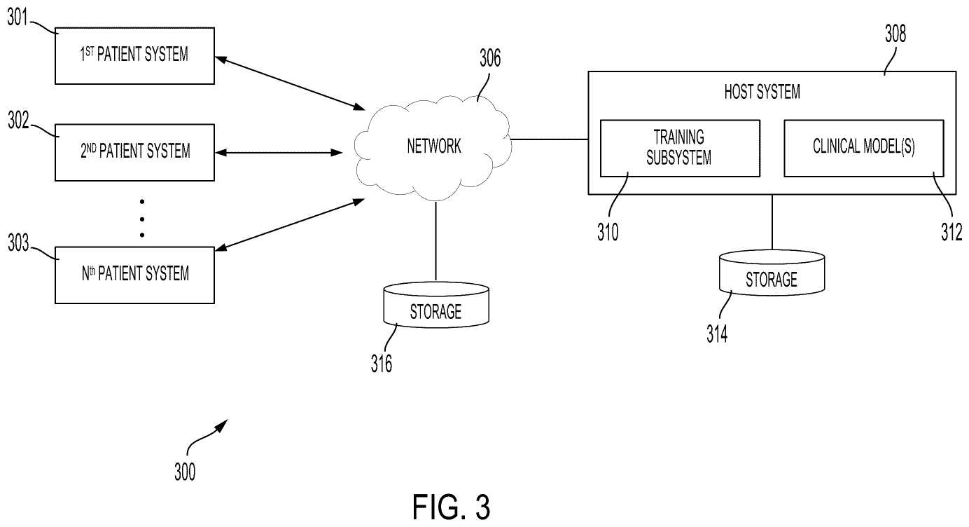

In some examples, the monitor 146 may be connected to a network-based host system (depicted in FIG. 3). For example, the communication connection(s) 148 or other elements of the monitor 146 may be capable of establishing connection to a computer network through any of a variety of wired or wireless communications protocols. In one example, the communication connection(s) 148 may be capable of connecting to a computer network via Wi-Fi protocol (or other wireless protocol). The computer network may include a local area network (LAN), wide area network (WAN), or other type of network. In some examples, the monitor 146 may be connected to the Internet.

As described herein, the monitor 146 may provide a wide variety of data associated with the example airway management system 100 to the network-based host system. For instance, the monitor 146 may provide data received from the acoustic sensor device 130, clinical data received from the ventilator 118 (e.g., data acquired by patient sensors 144 and/or ventilator sensors 126), patient data received from the ventilator 118, patient data provided by a clinician to the monitor 146, and/or other types of data to the host system. In further examples, the monitor 146 may provide data associated with the configuration of the example airway management system 100. For example, the configuration data may include the types of patient sensors 144 in use in the system 100 (e.g., pulse oximetry, capnometry, etc.), the configuration of the patient circuit 142 (e.g., single-limb or dual-limb), the types of sensor data provided by the ventilator sensors 126 (e.g., flow, pressure, etc.), and/or other configuration data. The network-based host system may use the received data to train one or more clinical models, which may be further used to provide a recommendation for one or more alert thresholds. In some examples, the host system may provide the trained clinical model(s) to the monitor 146, which may use the model during operation as described in further detail below.

In some examples, the ventilator 118 may be similarly capable of connecting and communicating with a network-based host system. For instance, the ventilator 118 may include the same or similar elements as the monitor 146, such as a processor, memory, communication connections, etc., that enable the ventilator to connect to the computer network and provide data to the host system. Similar to the above description, the ventilator 118 may provide sensor data, patient data, and/or other data to the host system, and may receive recommendations for clinically relevant alert thresholds and/or other types of data/information from the host system.

In an example scenario, during operation of the airway management system 100, the ventilator 118 provides inhaled breathing gases 120 to the patient 102 during inhalation and receives exhaled breathing gases during exhalation. The ventilator sensors 126 may acquire measurement data for flow, pressure, RR, and volume of the inhaled gas mixture 120 and the exhaled breathing gases. The patient sensors 144 may acquire SpO₂, ECG, EtCO₂, HR, and/or other data from the patient 102. Data acquired from the ventilator sensors 126 and patient sensors 144 may be provided by the ventilator 118 to the monitor 146 via the ventilator cable 160. The monitor 146 further receives acoustic data from the acoustic sensor device 130. The acoustic data may indicate the position of the tracheal tube 108 (e.g., relative to a baseline position), any obstructions that may be present in the tracheal tube 108, airway constriction in the vicinity of the tracheal tube distal tip, and/or other data associated with the tracheal tube 108. The monitor 146 may also have access to patient demographic data (e.g., age, weight, etc.), such as patient data provided to the monitor 146 and/or ventilator 118 by a clinician and/or retrieved from a medical database, such as an electronic health record (EHR) database. The monitor 146 may transmit the patient data, sensor data, acoustic data, and/or configuration data to a network-connected host system, which analyzes the data using one or more clinical models. In other examples, the model may be stored locally and executed on the monitor 146 and/or ventilator 118. In some examples, the host system may use the received data to train or update one or more clinical models.

The clinical models may provide recommended alert thresholds, that are clinically relevant to the specific patient, for acoustic data based on the data provided by the monitor 146. For instance, based on the patient type and/or characteristics, alert thresholds for tube movement and/or obstruction percentage may be generated from one or more clinical models. As one illustrative example, using one or more clinical models, the system may determine that, given the age and weight of a patient (among other factors), the monitor 146 should provide an alert for obstructions that exceed 52% of the inner diameter of the tracheal tube 108, which may be correlated with a 5% reduction in SpO₂. In some examples, the host system may also determine that such an obstruction may also be correlated with a 10% change in EtCO₂. These changes in SpO₂ and EtCO₂ may be associated with health risks in the patient's demographic (e.g., age, weight, and other factors). Accordingly, an obstruction alert threshold of 52% may be set by the monitor 146. The host system may, in some examples, also provide one or more of the clinical models to the monitor 146, which may use the clinical models to track correlation between the acoustic data and the clinical data as ongoing acoustic and sensor measurements are performed.

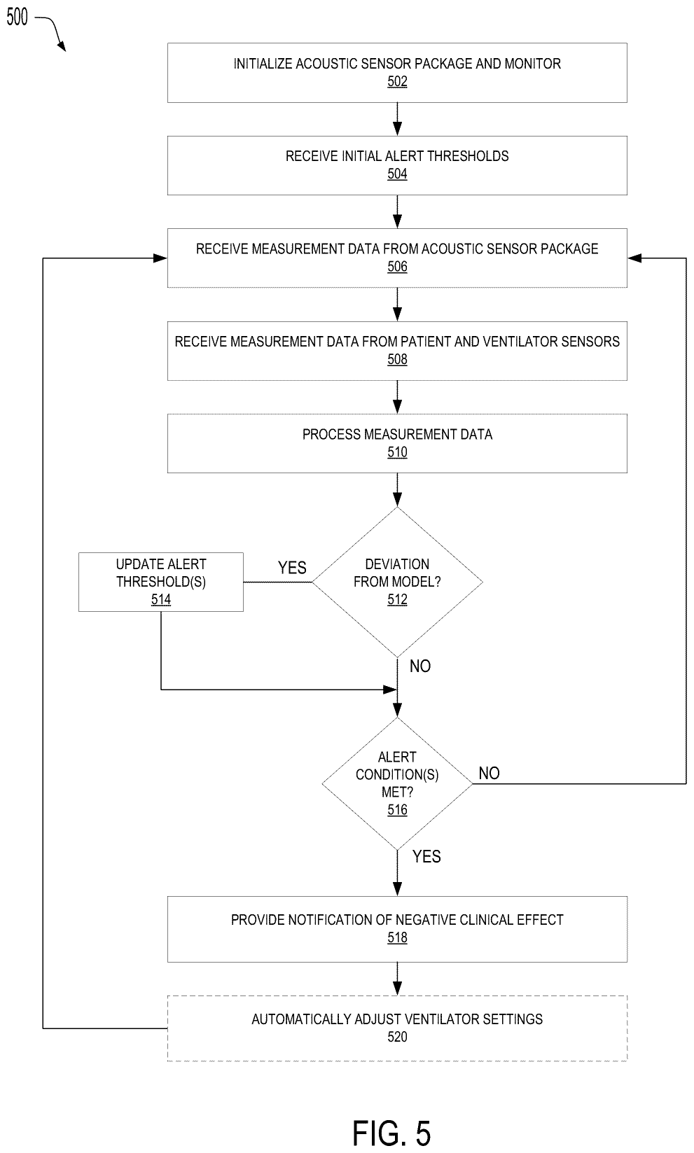

During continued operation of the airway management system 100, the monitor 146 may determine, based on the reflected acoustic pulses, that an obstruction in the tracheal tube 108 is present, and the obstruction exceeds 52% of the diameter of the tracheal tube 108. The monitor 146 may provide a notification or alert, such as a visual and/or audible alert, when the acoustic data indicates an obstruction larger than 52% in the tracheal tube 108. The ventilator 118 continues to provide SpO₂ and EtCo2 data to the monitor 146, which may use the data to further observe ongoing correlation with obstruction. That is, the monitor 146 may observe whether the SpO₂ and EtCO₂ data correlate with the level of obstruction predicted by the clinical model (e.g., 10%). If the trend of the SpO₂ and EtCO₂ data does not correlate with the trends predicted by the model(s), then the alert threshold may be adjusted based on the divergence from the predicted trend. For instance, if the SpO₂ for the particular patient has a trend that indicates the patient is more sensitive to obstructions than initially predicted by the model, the alert threshold may be adjusted to be more sensitive (e.g., a smaller percentage of obstruction). Additional discussion of dynamically adjusting thresholds is provided below with respect to at least FIG. 5. Variability between the clinical model and the observed data may be provided to the host system as feedback, such as to improve performance of the clinical model.

In some examples, rather than the ventilator 118 providing sensor and other data to the monitor 146, the monitor 146 may provide acoustic and other data to the ventilator 118. Accordingly, the ventilator 118 may connect to a network-based host system and may provide alerts based on alert thresholds suggested by the host system, as described above.

FIG. 2A depicts a view of an example acoustic sensor package or device 230, which may be similar to, or the same as, acoustic sensor device 130. The acoustic sensor device 230 includes a fitting 261 (e.g., a 15 mm fitting) for connecting tubing associated with a ventilator (such as ventilator 118 and patient circuit 142). The fitting 261 is connected to a sound-sensing section 262, which includes elements associated with the generation, detection, and processing of acoustic pulses. For example, the sound-sensing section 262 includes an acoustic generator (e.g., acoustic generator 132) and an acoustic sensor (e.g., acoustic sensor 134), among other elements. In some examples, the acoustic generator 132 may be housed within the fitting 261. The sound-sensing section 262 is further connected to a nozzle 263 which may couple to an ETT (e.g., tracheal tube 108).

The acoustic sensor device 230 includes an internal lumen that is continuous between a proximal opening 259 in the fitting 261 and a distal opening 265 in the nozzle 263. When tubing associated with a patient circuit is connected to the fitting 261 and an ETT is connected to the nozzle 263, inhaled breathing gases (e.g., gas mixture 120) may be provided to the patient (e.g., patient 102) from the ventilator. Inhaled breathing gases pass through the acoustic sensor device 230, from the proximal opening 259 to the distal opening 265. When the patient exhales through the ETT, the exhaled breathing gases pass through the acoustic sensor device 230 from distal opening 265 to the proximal opening 259.

During operation, the acoustic sensor (located in the sound-sensing section 262) may detect both transmitted and reflected acoustic pulses. The acoustic sensor, and/or elements associated with the acoustic sensor, provide acoustic-based signals that are based on the received pulses. The sound sensing elements generate electrical signals (e.g., the acoustic-based signals) that represent the acoustic signals. These acoustic signals consist of the received incident and reflected waves and are provided by the acoustic receiver and/or its associated elements. As depicted in FIG. 1 and described above, the acoustic-based signals may be provided to an associated monitor (e.g., monitor 146 or 246) via an interface cable (e.g., interface cable 158). In examples, the sound-sensing section 262 may include elements associated with a wireless connection to the associated monitor, such as Bluetooth, Wi-Fi, etc.

FIG. 2B depicts a front view of an example monitor 246 that receives acoustic-based signals from the acoustic sensor device 230. The monitor 246 may be similar to, or the same as, monitor 146 depicted in FIG. 1 and may include the same or similar elements. For example, the monitor 246 includes portions or elements of a user interface (e.g., user interface 156), such as soft keys 256A, navigation buttons 256B, and an alert silence or response button 256C. The monitor 246 further includes an alert indicator 256D and a speaker 256E, both of which may be associated with the provision of an alert or other type of notification to a clinician.

The monitor 246 includes a connection panel 248, which may include one or more ports for connecting cables between the monitor 246 and other components, systems, devices, etc. For example, the interface cable between the monitor 246 and the acoustic sensor device 230 may be connected to the monitor 246 at the connection panel 248. The monitor 246 may also be connected to other devices in an airway management system (e.g., system 100) via one or more cables that attach to the monitor 246 at the connection panel 248. For example, the monitor 246 and may be connected to a ventilator (e.g., ventilator 118) via a ventilator cable (e.g., ventilator cable 160), which attaches to the monitor 246 at the connection panel 248. Other devices in the airway management system (e.g., patient sensors 144) may similarly be connected to the monitor 246 via one or more cables that connect at the connection panel 248. In some examples, the monitor 246 may be connected to a computer network using a suitable networking cable (e.g., an ethernet cable) that connects to the monitor 246 at the connection panel 248.

The monitor 246 includes a display 254, which may be similar to, or the same as, display 154 described above. The display 254 includes one example of display content 266, which provides information to the clinician. In other examples, the display content 266 may be arranged differently than the depiction of FIG. 2B and may provide different information, such as depicted in FIGS. 2C-D.

The display content 266 includes information associated with the position of the ETT in the trachea of a patient, based on acoustic-based signals provided by the acoustic sensor device 230. In the example depicted in FIG. 2B, the display content 266 indicates an alert condition associated with the position of the distal tip of the ETT. For example, the display content 266 includes a baseline position indicator 268 that provides a numerical indication of a shift in the position of the ETT tip, relative to a baseline position established during an intubation procedure (or established at other times during operation of the monitor 246). As depicted in FIG. 2B, the baseline indicator 268 indicates that the tip of the ETT has shifted by 1.3 cm from baseline. Adjacent to the baseline indicator 268 are directional indicators 269-70 that indicate the direction of the shift from baseline position. As a result of a shift in the tip of the ETT, the baseline indicator 268 and one of the directional indicators 269-70 is illuminated to visually indicate the positional shift. In the example depicted, the baseline indicator 268 and low indicator 269 are illuminated in an alert pattern that indicates that the ETT has shifted to a position in the trachea that is too low (e.g., the position has crossed a movement threshold). The high indicator 270 is left unilluminated or is illuminated without an alert pattern. In conjunction with indicators 268-69 being illuminated with an alert pattern, the display content 266 may also include an alert message 272 indicating that the ETT is too low in the trachea. The monitor 246 may also provide an audible alert through the speaker 256E.

In some examples, the ETT tip may shift from its baseline position, but the shift may not exceed a threshold needed to trigger an alert. In such examples, the baseline indicator 268 and directional indicators 269-70 may not be illuminated with an alert pattern and/or may be illuminated with a pattern indicating a positional shift that does not exceed an alert threshold.

The threshold for triggering the alert may be provided by a network-based host system that may be in communication with the monitor 246, such as when the monitor 246 is connected to a computer network. As described, the monitor 246 may provide data to the host system, which may analyze the data using one or more clinical models to determine clinically relevant alert thresholds. In other examples, the models may be executed locally on the monitor 246 to generate the clinically relevant alert thresholds. For example, the alert threshold for a condition in which the ETT is too low in the patient's trachea may be correlated with a first clinical measure 274A, which in this example may correspond to a decrease in measured SpO₂ (e.g., as provided by a pulse oximeter). For instance, based on the clinical data provided by the monitor 246, one or more clinical models may be executed to determine that a shift in ETT position of more than 1 cm combined with a drop in SpO₂ of more than 10% (e.g., relative to a baseline reading) may be correlated with a health risk to the patient. Thus, a recommended alert threshold at 1 cm of ETT displacement may be generated.

The ETT position may also be correlated with a second clinical measure 274B, which in this example may correspond to an increase in measured EtCO₂ (e.g., as provided by a capnometry monitor). Similar to the first clinical measure 274A, based on data provided by the monitor 246, one or more clinical models of the host system may determine that a shift in ETT position of more than 1 cm is associated with a change in EtCO₂ of more than 20% (e.g., relative to a baseline reading), which may be correlated with a health risk to the patient. Thus, the host system may provide a recommendation for an alert threshold at 1 cm of ETT displacement.

Additionally or alternatively, the host system may provide clinically relevant alert thresholds that are based on, or otherwise associated with, other types of clinical data, such as HR, RR, flow, pressure, etc. Thus, the display content 266 may include first and second clinical measures 274A-B (and additional clinical measures) associated with these other types of clinical data.

In some examples, thresholds for a combination of parameters are adjusted. For instance, a shift in ETT may be combined with a change in passageway size to form a monitored combination parameter. As an example, a 1 cm shift of the ETT tube may result in little to no clinical effect with the ETT tip remains in the trachea. If, however, the 1 cm shift results in the ETT tip entering the mainstream bronchus, then there likely will be a clinical effect, such as a reduction in SpO2. In such examples, a shift in the ETT position is detected as well as a change in passageway size (e.g., a reduction in size due to the bronchial position).

An absolute delay of the in the airway echo within a search window may be another monitored parameter for which a clinically relevant threshold may be developed. For example, if the airway echo is far to the left in the search window, this would indicate a low ETT, and an echo far to the right would indicate a high ETT. Therefore, the relative position (or delay) in the window may be correlated with a clinical measure. For example, when the echo is far to the left (and near the carina) there may be an echo delay (from the left side of the window to the echo) where the SpO2 decreases (where the tip moves into the carina). The delay where this change occurs may be used to define an alert threshold.

In some examples, the monitor 246 may display a readout of the clinical measures 274A-B on the display 254. For instance, the clinical measures 274A-B may be continually displayed, such as in FIG. 2B, or may be displayed at the onset of an alert condition. In still other examples, the clinical measures 274A-B may not be displayed by the monitor 246.

In some examples, the generated clinically relevant thresholds may be implemented automatically. In other examples, the thresholds may be displayed as suggested or recommended thresholds. The clinician may then choose to accept (or reject) such recommended thresholds by interacting with the monitor 246, such as via buttons 256A-B.

FIGS. 2C-D depict additional examples of display content 266 associated with other examples of clinically relevant alert conditions. FIG. 2C depicts display content 266 for an example alert condition associated with an ETT obstruction. The display content 266 includes an ETT graphic 276, within which an obstruction indicator 277 is depicted. A depth gauge 280 is positioned adjacent to the ETT graphic 276. The depth gauge 280 includes depth markings that indicate distance relative to the tip of the ETT graphic 276. Acoustic data provided by the acoustic sensor device 230 may be analyzed to determine the location of the obstruction within the ETT, and the location of the obstruction indicator 277 may be placed according to the determined location. In the example, the obstruction indicator 277 is positioned at approximately 6 cm from the distal tip of the ETT graphic 276, which indicates that the obstruction is located approximately 6 cm from the distal tip of the actual ETT.

The acoustic reflections may also be analyzed to determine the severity of the obstruction, such as the percent occlusion of the obstruction. The display content 266 also includes an obstruction proportion indicator 278, which represents the proportion of the cross-sectional diameter or the diameter of the ETT occupied by the obstruction. In the example depicted, the obstruction occupies approximately 35% of the cross-sectional area of the ETT.

As described above for the ETT position alert condition (FIG. 2B), the monitor 246 may provide data for input to clinical models that use the data to recommend clinically relevant alert threshold for ETT obstruction. In the current examples, the monitor 246 may provide ETT dimensions 275, such as ETT diameter and length, to the clinical models, in addition to the other types of data described above. Clinical models may determine that an obstruction may be correlated with a first and second clinical measure 274A-B (e.g., SpO₂ and EtCO₂, respectively). For example, using a clinical model, the host system may determine that an obstruction of 52% may be associated with a 10% decrease in SpO₂ and/or a 20% change in EtCO₂ (relative to a baseline readings) for this particular patient. Thus, the host system may provide a recommendation for an alert threshold of more than 52% ETT obstruction.

In examples where the alert threshold(s) are exceeded, the display content 266 may include an alert message 272 indicating that a clinically significant obstruction has been detected. Additionally, the monitor 246 may provide additional notifications of the alert, including providing an audible alarm, illuminating an alert indicator 256D, and/or other methods for providing notification.

FIG. 2D depicts display content 266 for an example alert condition associated with the passageway size. The passageway may be any of these tubular structures in which the ETT tip may be located: the trachea, a mainstem bronchus, the supraglottic cavity, or the esophagus. The ETT graphic 276 may include a passageway graphic 282 positioned near and/or around the distal tip of the ETT graphic. The passageway graphic 282 provides a visual representation of the diameter (or cross-sectional area) of the passageway in the vicinity of the ETT tip. The display content 266 further includes a current diameter measure 284 and a baseline diameter measure 286.

Acoustic data provided by the acoustic sensor device 230 may be analyzed to determine the diameter of the passageway in the vicinity of the ETT tip following placement of the ETT during intubation (or at other times). This measurement may be included in the display content 266 as a baseline diameter 286. During use, the acoustic sensor device 230 and/or monitor 246 continues to perform ongoing measurements of the passageway diameter, and the display content 266 may provide a readout of the current diameter 284. As described above, using data provided by the monitor 246 and one or more clinical models, the host system may determine correlations between airway current diameter 284 and a first and/or second clinical measure 274A-B (e.g., SpO₂ and EtCO₂, respectively). The host system may use clinical models to further determine one or more clinically relevant alert thresholds that correspond to a health risk to the patient and provide these alert thresholds to the monitor 246.

For example, the clinical models may be used to determine that a reduction in airway diameter (e.g., a constriction) to less than 4.5 mm may be associated with a 10% decrease in SpO₂ and a 20% increase in EtCO₂ (relative to baseline readings). Thus, a recommendation for an alert threshold for a current diameter 284 of less than 4.5 mm may be provided. In examples where the alert threshold(s) are exceeded, the display content 266 may include an alert message 272 indicating that a clinically relevant constriction has been detected and may further provide additional audio and/or visual notifications.

FIG. 3 depicts a network-connected system 300 that includes a plurality of patient systems 301-303, a network-based storage 316, a host system 308, and local storage 314. The patient systems 301-303, network-based storage 316, and host system 308 communicate with each other over a network 306. The patient systems 301-303 represent a larger population of patient systems that may be connected to the host system 308 through the network 306. In some examples, the system 300 may include a very large population of patient systems that communicate with the host system 308 as described herein. Each of the patient systems 301-303 may be connected to different patients at different times, such as when a patient system 301-303 is disconnected from one patient, cleaned, and subsequently used with another patient. Further, in some examples the patient systems 301-303 may operate in different clinics, while in other examples two or more of the patient systems may be co-located.

The patient systems 301-303 are each representative examples of airway management systems (e.g., example airway management system 100). Each patient system 301-303 includes at least a monitor (e.g., acoustic monitor 146, 246), an acoustic sensor package (e.g., acoustic sensor device 130, 230), and, in some examples, a ventilator (e.g., ventilator 118), which is connected to a patient via a patient circuit (e.g., patient circuit 142). However, in some examples, the patient systems 301-303 may each be configured differently from one another and/or may include different features, functions, and/or elements than each other. As one example, the patient systems 301-303 may each include a collection of various patient sensors (e.g., patient sensors 144). The first patient system 301 may include an SpO₂ sensor, the second patient system 302 may include an SpO₂ sensor and a capnometry monitor, and the third patient system 303 may include neither an SpO₂ sensor nor a capnometry monitor.

Further, the elements of each patient system 301-303 may be configured to operate differently and/or may provide different functionality. For example, the first and second patient systems 301-302 may include a dual-limb patient circuit, where each ventilator manages both inhaled and exhaled breathing gases. The third patient system 303 may be configured with a single-limb patient circuit, where the ventilator provides breathing gases during inhalation, but does not manage exhaled breathing gases during exhalation. Thus, the patient systems 301-303 may be capable of providing sensor data associated with the inhaled breathing gases (e.g., from ventilator sensors 126), such as inhalation flow, pressure, etc. Whereas the first and second patient systems 301-302 may further be capable of providing sensor data associated with exhaled breathing gases. The patient systems 301-303 (and additional patient systems) may each include different types and numbers of patient sensors that collectively provide a range of clinical data from a population of patients.

As noted above, the patient systems 301-303 are connected to the host system 308 via network 306. The network 306 may include any of a variety of computer equipment suitable for providing connectivity between the patient systems 301-303, network-based storage 316, host system 308, and/or other resources connected to the network 306. For example, the network 306 may include servers, routers, switches, cables, interconnects, and/or other types of computer equipment for providing connectivity between devices attached to the network 306. The network 306 may be configured as a LAN, WAN (e.g., the Internet), and/or other type of network configuration. In some examples, the network 306 may include computer equipment suitable for providing wireless connections, such as through Wi-Fi and/or other wireless protocols. Thus, in some examples, the patient systems 301-303, host system 308, network-based storage 316, and other resources/devices may connect to the network 306 via a wired connection (e.g., Ethernet cable) or via wireless connection (e.g., Wi-Fi).

The host system 308 may include one or more computing devices, such as a type of computer and/or server. In examples where the host system 308 includes a plurality of computing devices, the computing devices may be co-located or may be distributed to different physical locations across the network 306. For example, the host system 308 may include a plurality of cloud servers or other type of computing devices arranged as part of one or more computing clusters, data centers, and/or other arrangements. Similarly, the network-based storage 316 may include any of a variety of co-located or distributed storage devices, such as rack-mounted hard disk drives and/or other forms of mass storage. In examples, the network-based storage 316 may be a type of network-attached storage (NAS), storage area network (SAN), database, and/or other type of storage arrangement. The network-based storage 316 may include computing devices (e.g., computers, servers, etc.) that provide access to the stored data.

The host system 308 also includes local storage 314, which may be located with, and directly accessible to, the host system 308. For example, the local storage 314 may include internal or external memory storage devices, such as one or more hard disk drives and/or other forms of data storage. The local storage 314 may be distributed and accessible to one or more of the computing devices associated with the host system 308.

During operation, the host system 308 receives and processes data from the patient systems 301-303. As described above, the data provided by each of the patient systems 301-303 may vary, depending on the configuration of each patient system 301-303, the types and number of sensors provided within each patient system 301-303, the type of ventilator connected to each patient, and other factors that are specific to each patient system 301-303 and the patients connected thereto.

To briefly summarize, data provided to the host system 308 by the patient systems 301-303 may include patient data, clinical data, configuration data, and/or acoustic sensor data. The patient demographic data may include the age, weight, height, gender, body mass index (BMI), health condition (e.g., COPD, heart condition, etc.) and/or other types of patient-specific data. The clinical data may include sensor measurement data associated with patient sensors and/or ventilator sensors. For example, the clinical data may include measurements of SpO₂ (e.g., from pulse oximetry), EtCO₂ (e.g., from capnometry), HR, RR, V_{T}, flow, pressures, and/or data from other types of sensors. The configuration data may include information associated with the devices and/or medical equipment available within each patient system 301-303. For example, the configuration data may include the type of patient circuit in use (single-limb or dual-limb), the capabilities of the ventilator, the types of patient sensors available, and the like. The acoustic sensor data may include processed acoustic sensor data and/or unprocessed, or minimally processed, acoustic sensor data. Examples of processed acoustic sensor data include the percentage of ETT obstruction, level of airway constriction, displacement of the ETT tube in the trachea, and/or other conditions associated with the ETT and airway of each patient. Examples of unprocessed or minimally processed acoustic sensor data include unfiltered acoustic-based signals acquired by an acoustic sensor, and/or other types of signals.

During a training period, the host system 308 may provide the data received from the patient systems 301-303 to a training subsystem 310, which may be a type of executable program code, such as software, software applications, utilities, programs, algorithms, firmware, and/or other type of computer instructions. The host system 308 may also provide other training data sets to the training subsystem 310. For example, the host system 308 may have access to, or be provided with, additional clinical data, such as data from one or more clinical studies, clinical databases, and/or data from other clinical sources. In some examples, the additional clinical data may be stored in network-based storage 316 and/or local storage 314.

The training subsystem 310 may aggregate the received data (including any additional clinical data provided to the host system 308), such as over a large population of patients and patient systems 301-303, and/or over a specified period of time. The aggregated data may be stored in network-based storage 316 and/or local storage 314. The training subsystem 310 may be configured to analyze the aggregated data to determine or identify correlations between variables within the aggregated data. For example, using the aggregated data, the training subsystem 310 may identify a correlation between the degree of ETT obstruction (and/or other measures from the acoustic data) and changes in measured SpO₂ (and/or other measures from the clinical data) for particular patients and/or ventilation settings. The training subsystem 310 may identify that patients having a first type of characteristics are more sensitive to obstructions than patients having a different or second type of characteristics. As an example, the more sensitive patient type may experience a drop in SpO₂ from smaller obstructions that the less sensitive patient type. For instance, a 30% obstruction may cause negative clinical effects (e.g., lowered SpO₂) for the more sensitive patient type, whereas the same 30% obstruction have little to no clinical effect for the less sensitive patient type.

In additional examples, the training subsystem may identify multiple correlations within the aggregated data. For example, the training subsystem 310 may identify that the degree of ETT obstruction is correlated with measured SpO₂, EtCO₂, and HR for different types of patients and/or different types of ventilation modes or settings. In still further examples, the training subsystem may identify correlations within the aggregated data between the acoustic sensor data, clinical data, and patient data. As another example, the training subsystem may identify a first correlation between ETT obstruction and SpO₂ for a first patient type or subset (e.g., male, age 45-50, 180-200 lbs) and may identify a second correlation between ETT obstruction and SpO₂ for a second patient type or subset (e.g., male, age 20-25, 180-200 lbs). For instance, the training subsystem 310 may identify that SpO₂ is more sensitive to ETT obstruction for the first cohort (e.g., increases in ETT obstructions result in larger changes in SpO₂), than for the second cohort (e.g., increases in ETT obstructions result in smaller changes in SpO₂). In other examples, the training subsystem 310 may find a plurality of correlations between variables provided in the aggregated data.

During the training period, the training subsystem 310 establishes (e.g., trains) one or more clinical models 312 using the identified correlations and the aggregated data. The training period may continue until a suitable amount of data has been aggregated to achieve a desired statistical significance (e.g., statistical power) and/or until the trained clinical models 312 meet desired performance criteria. Once the clinical models 312 are trained, the clinical models 312 use newly received data from a patient system 301-303 as input, and provide, as output, recommendations for one or more clinically relevant alert thresholds for acoustic sensor data collected by each patient system 301-303. The alert thresholds are generated for each patient system 301-303, based on the input data provided by each patient system 301-303 to the clinical models 312. The alert thresholds are provided to the corresponding patient system 301-303 by the host system 308 via the network 306. As described above, the clinically relevant alert thresholds may be used by a monitor (e.g., monitor 146, 246) to provide a notification to a clinician of a health risk detectable by the acoustic sensor package and monitor (e.g., ETT obstruction, airway constriction, etc.).

In examples, the clinical models 312 may include data distributions, probability distributions, statistical models, one or more model equations, equation coefficients, model parameters, and/or other statistical or mathematical elements suitable for providing clinically relevant alert threshold based on input data received from a patient system 301-303. In some examples, the clinical models may be machine learning (ML) and/or artificial intelligence (AI) models, such as neural networks and the like. Some example clinical models may include linear regression models, logistic regression models, decision trees, random forests, support vector machines (SVMs), and/or neural networks. Such models may be trained using supervised training techniques based on labeled training data that indicates the correlations between the acoustic data and the other conditions, such as patient type and/or ventilation parameters. In some examples, the clinical models may include or utilize models that are trained using unsupervised training methods. Such models are capable of identifying pattern in data that is unlabeled with a ground truth or known outcome variable, and such models may be used for clustering of the data and identifying associations. The models may include K-means clustering models, hierarchical clustering models, principal component analysis (PCA) models, and the like. Semi-supervised learning models may also be used for the clinical models 312. The clinical models 312 may be stored in network-based storage 316 and/or local storage 314, and accessed as needed by the host system 308, patient systems 301-303, and/or other resources/devices connected to the network 306.

The data aggregated from the patient systems 301-303 may be used in its received form as training data, particularly for unsupervised training implementations and models. In other examples, the data aggregated from the patient systems 301-303 may be further analyzed and processed to generate a training data set that may be used for supervised training. For instance, the aggregated data may be processed to positively identify situations where a negative clinical event or outcome occurred (e.g., clinically significant drop in SpO₂). The associated acoustic data (e.g., tube position, obstruction location, obstruction percentage) and the patient data, ventilator data, and/or sensor data for that event may then be labeled as a negative clinical event. Such labelled data may then be used for supervised training of the clinical models 312.

Following the training period, the host system may continue to provide newly received data from the patient systems 301-303 to the training subsystem 310. For example, newly received data be used by the training subsystem 310 to improve the statistical significance of distributions and/or statistical models used by the training subsystem 310 and/or to improve performance of one or more of the clinical models 312. In some examples, the training subsystem 310 may update or revise the clinical models based on the newly received data through reinforcement learning.

The population of patient systems 301-303 that provide input data to the clinical models 312 following the training period may be different than the population of patient systems that provide training data to the training subsystem 310 during the training period. Over time, new patient systems may be added or removed from the population of patient systems 301-303 connected to the host system 308. In some examples, the patient systems 301-303 that provide input data to the clinical models 312 following training may be the same patient systems 301-303 that provided training data during the training period. Additionally, patients connected to the patient systems 301-303 following the training period may different than the patients connected to the patient systems 301-303 during the training period.

As described, the clinical models 312 provide, as output, one or more recommendations for clinically relevant alert thresholds for acoustic data processed by a monitor (e.g., 146, 246) within each patient system 301-303. To determine clinically relevant alert thresholds, the clinical models 312 may utilize risk data, which may include data that relates one or more clinical data measures (e.g., SpO₂, EtCO₂, etc.) with one or more health risks. For example, risk data for SpO₂ for an otherwise healthy adult patient may indicate a normal oxygen saturation level in the range of 95%-98%, a below normal oxygen saturation level in the range of 92%-95%, and hypoxia for oxygen saturation levels below 92%. In another example, the risk data for SpO₂ for a COPD patient may indicate a normal oxygen saturation level in the range of 90%-92%, below normal oxygen saturation level in the 88%-92%, and hypoxia for oxygen saturation levels below 88%. In other examples, the risk data may associate other types of clinical data (e.g., EtCO₂, HR, etc.) and/or patient demographic data (e.g., age, weight, etc.) with other established health risks.

Additionally, the risk data may indicate health risks based on various measures provided in the clinical data. For example, the risk data may indicate a health risk based on an absolute measure, such as in the example above where risk is categorized by an absolute level of SpO₂. In other examples, the risk data may indicate health risk based on changes in a clinical measure, such as a drop in the absolute level of SpO₂ (e.g., a drop of 2%) or a percent change in the level of SpO₂ (e.g., a 10% decrease in SpO₂). In still other examples, the risk data may indicate health risks for other measures or changes in measures of clinical data.

The risk data may be made available to the host system 308 by a variety of methods. In some examples, the risk data may be provided to the host system 308 through another network connected resource (not depicted), such as through another computing device, server, etc. The risk data may be provided directly to the host system 308 and/or may be provided to network-based storage 316 and/or local storage 314 and accessed by the host system 308. The risk data may be based on established health data, data trends, clinical studies, healthcare practice, and/or by other methods for establishing health risk. The risk data may be provided in the form of data distributions, statistical distributions, risk categories, data tables, and/or other forms for providing data. In some examples, the risk data may be used by the training subsystem 310 during the training period.

In a first example scenario, the first patient system 301 may provide data to the host system 308 for input to one or more trained clinical models 312. In this example, the received data includes patient data, such as the gender, age, and weight of a patient receiving breathing assistance from a ventilator of the first patient system 301. The received data also includes acoustic sensor data, such as ETT tube dimensions (e.g., tube dimensions 275) and baseline airway diameter (e.g., baseline diameter 286). For the patient data provided and using risk data for the patient's cohort, the clinical models 312 may determine that a 10% decrease in the patient's SpO₂ level and a 20% change in the patient's EtCO₂ level may pose a health risk to the patient. The clinical models 312 may further determine that an ETT obstruction greater than 52% may bring about these changes in SpO₂ and EtCO₂. In addition, the clinical models 312 may determine that if the patient's airway constricts to less than 4.5 mm, the patient may further experience the above changes in SpO₂ and EtCO₂. Thus, the clinical models 312 may recommend a clinically relevant alert threshold of 52% for ETT obstruction and 4.5 mm for airway constriction. The host system 308 may provide these recommendations to the first patient system 301, which may apply the clinically relevant thresholds to processed acoustic sensor data. ETT obstructions greater than 52%, and airway constrictions less than 4.5 mm, may each trigger a notification to the clinician.

In a second example scenario, the second patient system 302 may provide data to the host system 308 and receive recommendations for clinically relevant alert thresholds from one or more clinical models 312, as described in the first example scenario. The second patient system 302 may also be provided with a local instance of one or more of the trained clinical models 312 and any data that may support the clinical models (e.g., risk data, clinical data distributions, etc.). A monitor of the second patient system may use the clinical models 312 to observe conditions where clinical and/or acoustic sensor data deviates from the modeled behavior (e.g., the data may not correlate as predicted by the clinical models 312). For instance, a monitor of the second patient system 302 may actively receive clinical data from an associated ventilator, such as ongoing SpO₂ and EtCO₂ measurement data acquired by patient sensors. The monitor may observe an ETT obstruction greater than 52% but may further observe that SpO₂ has decreased by only 6% and that EtCO₂ has increased by only 8%. Rather than providing a notification per the recommended alert threshold, the monitor may update the ETT obstruction alert threshold and continue to monitor the clinical and acoustic data. For example, the monitor may update the alert threshold to ETT obstructions greater than 54% and continue to observe acoustic sensor and clinical data.

Further, data may be provided to the host system 308 so that the clinical models 312 may be updated, if necessary. For example, the second patient system 302 may provide updated clinical data (e.g., SpO₂ and EtCO₂ measurement data showing the deviation) and corresponding acoustic sensor data to the training subsystem 310, which may use the data to revise one or more clinical models 312. Additionally or alternatively, the monitor of the second patient system 302 may update a local instance of one or more of the clinical models 312, so that data collected from the patient may be used to provide a revised clinically relevant alert threshold. For instance, the monitor may collect additional clinical and/or acoustic data to retrain the local instance of one or more clinical models 312.

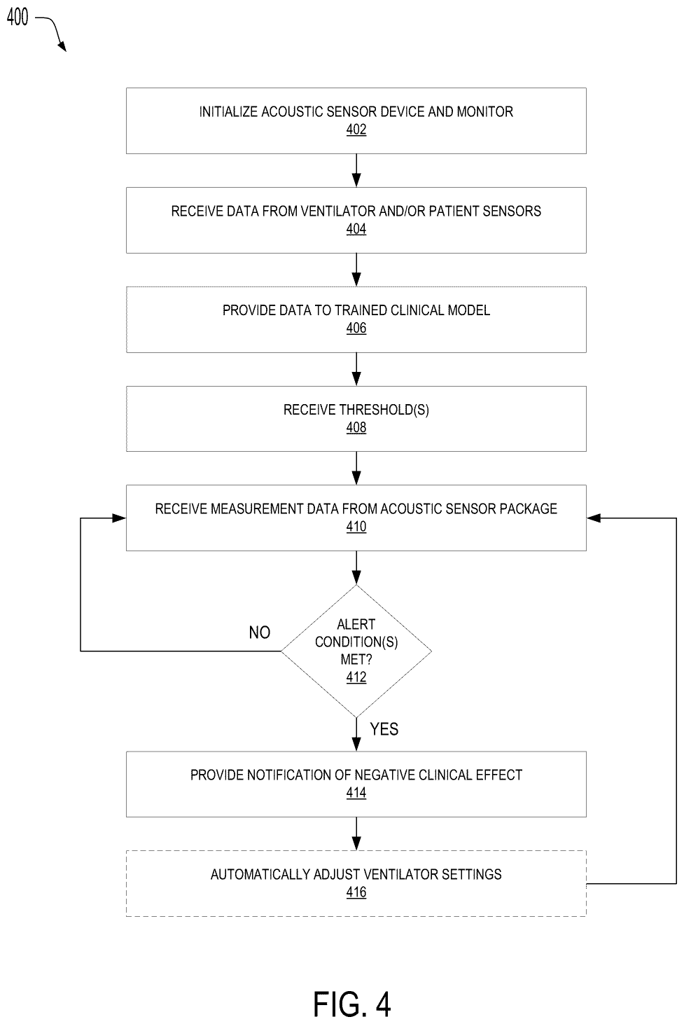

Example methods 400 (FIG. 4) and 500 (FIG. 5) are now presented, which may provide additional details associated with the first and second example scenarios described above, respectively.

FIG. 4 depicts an example method 400 for providing a notification of reduced respiration based on one or more clinically relevant alert thresholds. The thresholds are applied to data acquired by an acoustic sensor device (e.g., acoustic sensor device 130, 230) and processed by an associated monitor (e.g., monitor 146, 246). While operations of example method 400 are described below as being performed by the monitor, some or all of example method 400 may be performed by other devices or systems associated with an airway management system (e.g., example airway management system 100). For example, some or all of method 400 may be performed by the monitor, ventilator (e.g., ventilator 118), other suitable equipment that may be part of the airway management system, or a combination thereof. The monitor is connected to an acoustic sensor device (e.g., acoustic sensor device 130, 230) that provides acoustic-based signals to the monitor as described with respect to FIG. 1. The ventilator may include a plurality of ventilator sensors (e.g., ventilator sensors 126) and may be connected to a plurality of patient sensors (e.g., patient sensors 144). In some examples, one or more patient sensors may be connected to the monitor.

At operation 402, the monitor and acoustic sensor device are initialized. The initialization may include powering-on the monitor and acoustic sensor package, establishing communication between the monitor and acoustic sensor package, calibration of instrumentation, and the like. During initialization, the acoustic sensor package may collect baseline measurements and provide the corresponding measurement data to the monitor. For example, the acoustic sensor package may provide data indicating a baseline position of the ETT, the airway diameter in the vicinity of the ETT tip, and/or other baseline information associated with the ETT.

Initialization may further include establishing communication with devices that are communicatively coupled to the monitor, such as the ventilator. In examples where one or more patient sensors are connected to the monitor, communication may be established between the monitor and sensor(s).

The initialization may include receiving user inputs from a clinician or other user to configure the monitor for operation, or to configure devices connected to the monitor, such as the acoustic sensor package and/or ventilator. Such configuration may include setting operating ranges, enabling sensor features or functions, setting circuit operating parameters (such as programmable filtering or amplification), selecting sampling rates, data rates, or setting other selectable operating features provided by the monitor and/or acoustic sensor package. In some examples, the initialization may occur before or after the patient is intubated and a complete breathing circuit is established.

Initialization may also include receiving patient demographic data. As described above, patient demographic data may include the age, weight, health condition, etc. of the patient. The patient data may be provided to the monitor by clinician input, such as through a user interface of the monitor. In other examples, the patient data may be input to a ventilator, and the ventilator may provide the patient data to the monitor. For instance, the ventilator may be connected to the monitor (e.g., by ventilator cable 160) and may provide the patient data via the connection. In further examples, the monitor may receive patient data from a computing device associated with an electronic medical or health record system.

In addition, during initialization, the monitor may connect to a network-based host system (e.g., host system 308). For example, the monitor may establish communication with the host system through a wired (e.g., Ethernet) or wireless (e.g., Wi-Fi) connection to a computing network, to which the host system may also be connected.

At operation 404, data is received from the ventilator and/or connected patient sensors. The ventilator data may include data from ventilator sensors, such as flow sensors, pressure sensors, and/or other types of sensors that measure ventilation parameters. The data may include data derived from ventilation sensor measurements, such as inhalation and/or exhalation volume data that is derived from flow measurements. The ventilator data may also include data regarding the particular ventilation mode and/or ventilation settings that is currently active for providing the ventilation to the patient.

The patient sensor data that is received includes data acquired by the connected patient sensors, such as a pulse oximeter, capnometry monitor, HR monitor, etc. The data may include SpO₂, EtCO₂, HR, RR, and/or other data acquired by patient sensors. In some examples, the ventilator data and patient data may continue to be received throughout the performance of method 400. As such, trend data and/or changes in the received data may be used by the clinical models. In some examples, the trend or change data may be calculated before being processed

At operation 406, data received at operation 404 is provided to one or more trained clinical models (e.g., clinical models 312). The provided data may include patient data, ventilator sensor data, patient sensor data, and/or configuration data, as described above. In some examples, the clinical model(s) are hosted locally at the monitor and/or the ventilator. In such examples, providing the data to the clinical model(s) includes providing the data as a local input to the model(s). In other examples, the clinical model(s) are executed remotely from the monitor, such as at host system or server. In such examples, providing the data to the clinical model(s) includes transmitting the data to the remote system, such as via a network.

At operation 408, based on the processing of the input data by the clinical model(s), the monitor receives one or more clinically relevant thresholds that may be applied to newly received acoustic sensor data. The thresholds may include one or more thresholds used to evaluate clinically relevant changes in ETT tube position, ETT obstruction, airway constriction, and/or other conditions or states of the ETT. The clinically relevant thresholds may include one or more absolute thresholds. For example, the host system may provide an absolute threshold for ETT obstruction at a level of 52% (e.g., ETT obstructions larger than 52% exceed the absolute threshold). In other examples, the host system may provide other types of absolute thresholds, such as a passageway diameter or cross-sectional area threshold (which may be a percentage or function of the ETT size/diameter), an ETT tip position threshold (e.g., the absolute position of the ETT within the trachea).

In further examples, the clinically relevant thresholds may include one or more relative thresholds. As an example, one of the relative thresholds may be based on changes in ETT tube position relative to a baseline position. As another example, one of the relative thresholds may be based on changes in airway diameter in the vicinity of the ETT tip relative to a baseline diameter. In some examples the relative thresholds may be based on baseline acoustic measurements acquired during initialization (e.g., at operation 402).

In examples where the one or more clinical models are executed locally, the clinically relevant threshold(s) may be received as output from the clinical model(s). In examples where the one or more clinical models are executed remotely, the clinically relevant threshold(s) may be received from the remote system, such as via the same network through which the input data was transmitted.

At operation 410, acoustic sensor data is received from the acoustic sensor device. The acoustic sensor data may also be referred to as acoustic reflectometry data. The acoustic reflectometry data may be in the form of unprocessed, or minimally processed, acoustic-based signals that are further processed by the monitor. In some examples, the acoustic sensor package may process the acoustic-based signals and provide the processed data to the monitor. The processed acoustic data may include data associated with ETT position in the trachea, ETT obstruction, passageway size around the ETT tip, and/or other types of data. For instance, as part of operation 410, the monitor may process the acoustic reflectometry data to determine one or more values for the acoustically measurable parameters, such as ETT position, ETT movement, ETT obstruction position and/or size, airway constriction (e.g., airway diameter distal to the ETT), among other parameters measurable by the acoustic reflectometry system.

At operation 412, elements of the monitor (e.g., processor 150) compare the processed acoustic reflectometry data to one or more clinically relevant thresholds provided by the host system. In examples where the acoustic measurement data does not indicate measurements that exceed one or more of the clinically relevant thresholds, flow proceeds “NO,” and the monitor may continue to receive measurement data from the acoustic sensor device as it is acquired and/or processed.

In examples, where the acoustic measurement data includes one or more measurements that exceed one or more clinically relevant thresholds, flow proceeds “YES” to operation 414. At operation 414, the monitor provides an alert or notification that a condition is occurring that may be causing, or soon to cause, a negative clinical effect for the current patient. The notification may be provided in the form of an audio and/or visual alert. For example, the monitor may provide an audible alarm through a speaker included with the monitor (e.g., speaker 256E). Additionally or alternatively, the monitor may provide one or more forms of visual alert. In one example, the monitor may illuminate a visual alert indicator, such as a light source, on the exterior of the monitor (e.g., alert indicator 256D). In other examples, the monitor may provide the visual alert through a display (e.g., display 154). For instance, the monitor may illuminate one or more graphics of the display in an alert pattern or color, may cause graphics of the display to flash or blink, and/or may provide other forms of visual alert notification.

In response to the notification, the clinician may take action to improve respiration. For example, where the alert is related to an obstruction in the ETT, the clinician may apply suction within the ETT to remove a clinically relevant obstruction. In other examples, the clinician may reposition the ETT and/or adjust ventilator settings to improve respiration of the patient.

At operation 416, in some instances, the monitor may be configured to automatically provide control signals to the ventilator to adjust one or more ventilator settings or generate one or more recommendations for adjustments to one or more ventilator settings. The adjustments may include control of pressure, flow, and/or volume for the inhalation and/or exhalation phases of breathing, among other possible adjustments. For example, acoustic sensor data may indicate an airway constriction during an end portion of exhalation, where the constriction exceeds a clinically relevant threshold for airway diameter. Having detected the alert condition at operation 412 and provided a notification to the clinician at operation 414, the monitor may adjust one or more settings of the ventilator to prevent the airway constriction. In one example, the monitor may adjust the ventilator setting for positive end-expiratory pressure (PEEP), to maintain airway patency near the end of exhalation. In other examples, the monitor may automatically provide control signals to the ventilator to adjust other ventilator settings.

The clinician may configure the monitor and/or ventilator to enable automatic adjustments and to provide settings for managing the automatic adjustments. For example, the clinician may enable automatic adjustments for some ventilator settings (e.g., pressure and flow) but may not enable automatic adjustments for other settings (e.g., volume). In some examples, some ventilator settings may be automatically adjustable depending on the operating mode of the ventilator (e.g., volume assist, pressure assist, etc.). In additional examples, the clinician may configure the range over which the monitor may automatically adjust the pressure, flow, volume, and/or other setting of the ventilator. For the example given above, the clinician may provide a range over which the PEEP setting may be automatically adjusted.

The automatic adjustments may be configured and enabled during operation of the monitor and/or ventilator, such as while ventilation is being provided to the patient. In some examples, automatic adjustments may be configured during initialization, such as during operation 402.

Following the provision of a notification to the clinician at operation 414 and any automatic adjustment of ventilator settings (if enabled) at operation 416, the monitor continues to receive and process measurement data from the acoustic sensor package. The monitor may again provide notification (or continue to provide notification) of an alert condition in which one or more acoustic measurements exceed one or more clinically relevant alert thresholds.

Operations 404-408 of method 400 may also repeat periodically. For instance, as the sensor data updates or changes over time, new or different clinically relevant thresholds for the acoustic sensor data may be need or be more relevant. Such updating may occur at set time intervals (e.g., every hour) and/or upon one or more of the patient sensor signals diverging from a baseline by a threshold amount.

In examples, the monitor, ventilator, and/or other devices that may be part of the airway management system may provide additional data to the host system to improve performance of one or more of the clinical models. For example, updated clinical data, acoustic sensor data, and/or other data may be provided to the host system to improve clinical model training, statistics, and/or to improve other metrics associated with the clinical models. The data may be provided at any point during operation of the airway management system, such as at any operation performed as part of example method 400.

FIG. 5 depicts another example method 500 for receiving, updating, and using clinically relevant alert thresholds. In method 500, the clinically relevant thresholds may be more specific to the particular patient that is being ventilated. For example, the actual effect of conditions measurable from the acoustic sensor data (e.g., obstruction, ETT movement, ETT position) on the patient may be actively compared to the expected effects predicted by the one or more clinical models. If the actual effect deviates from the predicted effect, the clinically relevant thresholds may be adjusted.

As described for example method 400, while the operations of example method 500 are described below as being performed by the monitor, some or all of example method 500 may be performed by other devices or systems associated with an airway management system. For example, some or all of method 500 may be performed by the monitor, ventilator, other suitable equipment that may be part of the airway management system, or a combination thereof

At operation 502, the monitor and acoustic sensor package are initialized. The initialization may be similar to, or the same as, the initialization described for operation 402 of method 400. For instance, initialization may include powering on the monitor and acoustic sensor package and establishing communication between the two devices. The initialization may further include establishing communication between the ventilator and monitor, and between the monitor and any patient sensors that may be connected to the patient (if applicable). In addition, the monitor may connect with a network-based host system, as described above.

At operation 504, the monitor receives initial clinically relevant alert thresholds, which may be provide by trained clinical models of the host system, as described for example method 400. For example, the monitor may provide patient data, clinical data, system configuration data, acoustic sensor data (e.g., baseline measurement data), and/or other types of data to the for input to the clinical model(s). The provided data may be input to one or more clinical models that output recommendations for alert thresholds for newly received acoustic sensor data.

Further, as part of receiving initial alert thresholds, the monitor may receive the clinical models used to generate the clinically relevant alert thresholds. For example, the monitor may receive one or more statistical models, data distributions, equations, coefficients, risk data, etc., that form the clinical models, and may execute local instances of the clinical models during operation (e.g., utilizing a processor). The clinical models may indicate correlations of how changes to one or more of the conditions measurable from the acoustic reflectometry data (e.g., obstructions, ETT movement) affect measurable patient parameters, such as how obstruction size affects SpO₂ levels.

In some examples, a clinician may provide the initial alert thresholds. For example, a clinician may set initial alert thresholds based on the patient's age, weight, respiratory condition, and/or any number of potential clinical factors associated with the patient receiving ventilation. In one example, the clinician may set a lower threshold for ETT obstruction for a COPD patient (e.g., 25% obstruction) compared to a patient without COPD (e.g., 30% obstruction). In other examples, the clinician may provide one or more alert thresholds based on other factors, such as experience with similar patients, preference, and/or other factors.

At operation 506, the monitor receives measurement data from the acoustic sensor package. As described for operation 410, the acoustic measurement data may include data associated with the acoustically measurable parameters, such as parameters such as ETT position, ETT movement, ETT obstruction position and/or size, airway constriction (e.g., airway diameter distal to the ETT), among other parameters measurable by the acoustic reflectometry system. At operation 508, the monitor may receive measurement data from patient sensors and/or ventilator sensors, such as the types of data described above (e.g., SpO₂, EtCO₂, HR, RR, pressure data, flow data, etc.).

At operation 510, the monitor processes the received measurement data. For example, the monitor may compare the received patient sensor and ventilator sensor data to patient population data (e.g., patient data distributions) included with the clinical models to determine whether the patient data deviates from the patient population data. In some examples, the monitor may analyze the received data to determine whether the received data correlates with the acoustic measurement data, in accordance with the clinical models. For example, a determination may be made based on the patient sensor data, the ventilator sensor data, and/or the acoustic sensor data acquired over time to determine if the patient is responding as expected or predicted by the clinical model. For instance, as an increase in the size of an obstruction occurs, a determination may be made as to whether the SpO₂ of the patient is changing (e.g., dropping) as predicted by the clinical model(s). Accordingly, the comparison may be between a determined value or values for the acoustically measurable parameter(s) to the patient sensor data to determine whether there is a divergences from the predicted correlation from the clinical model(s).

At operation 512, the monitor determines whether the relationship between patient sensor data, ventilator sensor data, and/or acoustic measurement data deviates from one or more clinical models. For example, the monitor may determine that the correlation between SpO₂ data and ETT obstruction is outside of an acceptable statistical range. In another example, the monitor may determine that the correlation between the SpO₂ data and ETT obstruction is more strongly or weakly correlated than indicated by the clinical models. For instance, a clinical model may indicate that an ETT obstruction of 52% is correlated with a 10% decrease in SpO₂. However, based on received measurement data, the monitor may determine that an ETT obstruction of 52% is correlated with only a 5% decrease in SpO₂. In other examples, the monitor may otherwise determine that the relationship between the received data is not accurately represented by one or more of the clinical models.

In examples where the monitor determines that the relationship between received data from patient sensors, ventilator sensors, and/or acoustic sensor package deviates from the provided clinical models, flow proceeds “YES” to operation 514. At operation 514, the monitor may update one or more clinically relevant alert thresholds. The monitor may use any of a variety of methods for determining the updated threshold(s). For example, the monitor may increase or decrease one or more clinically relevant thresholds by a specified amount. In the example above, the monitor may increase the alert threshold for ETT obstruction by a set amount each time a deviation is detected (e.g., 2%), so that the updated ETT obstruction threshold increases or decreases by the set amount (e.g., to become 34%). In other examples, the alert threshold may change proportionally based on the amount of

In other examples, the monitor may use a statistical method for determining how to update one or more clinically relevant thresholds. For instance, the monitor may analyze data received from the patient sensors, ventilator sensors, and/or acoustic sensor package to determine a new or corrected correlation between the received data. The monitor may use the updated correlation to further determine a new clinically relevant alert threshold. In still other examples, the monitor may utilize other methods for updating the alert threshold(s). Flow then proceeds to operation 516.

In examples where the monitor determines (at operation 512) that the relationship between received data from patient sensors, ventilator sensors, and/or acoustic sensor package does not deviate from the provided clinical models, flow proceeds “NO” to operation 516.

At operation 516, elements of the monitor compare the received acoustic measurement data to one or more clinically relevant thresholds generated from the clinical model(s), including any thresholds updated at operation 514. In examples where the acoustic measurement data does not indicate measurements that exceed one or more of the clinically relevant thresholds, flow proceeds “NO,” and the monitor may continue to receive measurement data from the acoustic sensor package, ventilator sensors, and/or patient sensors as it is acquired.

In examples where the acoustic measurement data includes one or more measurements that exceed one or more clinically relevant thresholds, flow proceeds “YES” to operation 518. At operation 518, the monitor provides an alert or notification that a condition is occurring that may be causing, or soon to cause, a negative clinical effect for the current patient. The notification may be provided in the form of an audio and/or visual alert, as described above for operation 414 of example method 400. In response to the alert, the clinician may adjust ventilator settings, adjust the position of the ETT, remove obstructions, and/or take other actions according to the type of alert.

At operation 520, in some instances, the monitor may be configured to automatically provide control signals to the ventilator to adjust one or more ventilator settings. The adjustments may include control of pressure, flow, and/or volume for the inhalation and/or exhalation phases of breathing, among other possible adjustments. The adjustments may alternatively be displayed or otherwise presented to the clinician as a recommendation that can be accepted to denied by the clinician. Methods for automatic adjustment of ventilator settings were also described above, with respect to operation 416 of example method 400.

Following the provision of a notification to the clinician at operation 518 and any automatic adjustment of ventilator settings (if enabled) at operation 520, the monitor continues to receive and process measurement data from the acoustic sensor package, ventilator sensors, and/or patient sensors. The monitor may again provide notification (or continue to provide notification) of an alert condition in which one or more acoustic measurements exceed one or more clinically relevant alert thresholds, including one or more updated alert thresholds.

In examples, the monitor, ventilator, and/or other devices that may be part of the airway management system may provide additional data to the host system to improve performance of one or more of the clinical models. For example, updated clinical data, acoustic sensor data, and/or other data may be provided to the host system to improve clinical model training, statistics, and/or to improve other metrics associated with the clinical models. In addition, updated alert threshold data, correlation data, and/or other data updated as a result of deviation from the clinical models may also be provided to the host system. The data may be provided at any point during operation of the airway management system, such as at any operation performed as part of example method 500.

Those skilled in the art will recognize that the methods and systems of the present disclosure may be implemented in many manners and as such are not to be limited by the foregoing aspects and examples. In other words, functional elements being performed by a single or multiple components. In this regard, any number of the features of the different aspects described herein may be combined into single or multiple aspects, and alternate aspects having fewer than or more than all of the features herein described are possible. Functionality may also be, in whole or in part, distributed among multiple components, in manners now known or to become known.

Furthermore, those skilled in the art will recognize that boundaries between the functionality of the above-described operations are merely illustrative. The functionality of multiple operations may be combined into a single operation, and/or the functionality of a single operation may be distributed in additional operations. Some operations may be omitted. Moreover, alternative embodiments may include multiple instances of a particular operation, and the order of operations may be altered in various other embodiments.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about” or “substantially” convey in light of the measurement techniques utilized herein. To the extent such terms may not be clearly defined or understood by one having skill in the art, the term “about” shall mean plus or minus ten percent.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

What is claimed is:

1. An acoustic reflectometry system comprising:
an acoustic sensor comprising:
a generator that emits acoustic pulses; and
a sensor that detects the emitted acoustic pulses and reflections thereof;
a monitor, in communication with the acoustic sensor, the monitor comprising:
a processor; and
memory storing instructions that, when executed by the processor, causes the acoustic monitor to perform operations comprising:
receive patient data for a current patient receiving ventilation;
provide the patient data as input to a trained clinical model;
receive, as output from the trained clinical model, a clinically relevant threshold for an acoustically measurable parameter;
process the detected incident and reflected waves to determine a value of the acoustically measurable parameter;
compare the value of the acoustically measurable parameter to the clinically relevant threshold; and
based on the comparison, generate a notification.

2. The acoustic reflectometry system of claim 1, wherein the patient data includes at least one of age, height, weight, or a known medical condition.

3. The acoustic reflectometry system of claim 1, wherein the acoustically measurable parameter is one of tracheal tube movement, tracheal tube position, obstruction size, or passageway size.

4. The acoustic reflectometry system of claim 1, wherein the operations further comprise:
receiving patient sensor data; and
providing the patient sensor data as input to the one or more trained clinical models, wherein the clinically relevant threshold is further based on the patient sensor data.

5. The acoustic reflectometry system of claim 4, wherein the patient sensor data includes at least one of capnometry data, pulse oximetry data, or electrocardiogram data.

6. The acoustic reflectometry system of claim 1, wherein the operations further comprise:
receiving ventilator data; and
providing the ventilator data as input to the one or more trained clinical models, wherein the clinically relevant threshold is further based on the ventilator data.

7. The acoustic reflectometry system of claim 6, wherein the ventilator data includes at least one of a current ventilation mode, a ventilation setting, a pressure value, a flow value, a respiratory rate, work of breathing, indication of patient-initiated spontaneous breaths.

8. A method for detecting clinical conditions with an acoustic system, the method comprising:
receiving acoustic data from an acoustic sensor coupled to a tracheal tube of a patient being ventilated;
receiving patient demographic data for the patient;
receiving patient sensor data;
processing the acoustic data to determine a value for an acoustically measurable parameter;
based on at least the patient demographic data, receiving from a clinical model a predicted correlation between the patient sensor data and the acoustically measurable parameter;
based on the value of the acoustically measurable parameter and the patient sensor data, determining that the patient sensor data and the acoustically measurable parameter diverge from the predicted correlation; and
based on the divergence, adjusting a threshold for the acoustically measurable parameter.

9. The method of claim 8, wherein the acoustically measurable parameter is one of tracheal tube movement, tracheal tube position, obstruction size, or passageway size.

10. The method of claim 9, wherein the acoustically measurable parameter is obstruction size and the patient sensor data is pulse oximetry data.

11. The method of claim 10, wherein the divergence indicates that patient is more sensitive to obstruction size, and adjusting the threshold includes reducing the threshold.

12. The method of claim 8, wherein the patient sensor data is at least one of capnometry data, pulse oximetry data, or electrocardiogram data.

13. The method of claim 8, further comprising:
providing the patient demographic data as input to the clinical model; and
receiving, based on processing of the patient demographic data by the clinical model, an initial clinically relevant threshold for the acoustically measurable parameter,
wherein adjusting the threshold includes adjusting the initial clinically relevant threshold.

14. The method of claim 8, further comprising:
determining that a subsequent value for the acoustically measurable parameter exceeds the adjusted threshold; and
generating a notification based on determining that the value exceeds the adjusted threshold.

15. The method of claim 8, wherein the patient demographic data includes at least one of age, height, weight, or a known medical condition.
